(19) United States
(12) Patent Application Publication
(10) Pub. No.: US 2006/0052800 A1
(43) Pub. Date: Mar. 9, 2006

(54) Surgical tool for incontinence sling implantation

(75) Inventor: E. Skott Greenhalgh, Wyndmoor, PA (US)

Correspondence Address: SYNNESTVEDT & LECHNER, LLP, 2600 ARAMARK TOWER, 1101 MARKET STREET, PHILADELPHIA, PA, 191072950, US

(73) Assignee: Secant Medical, LLC, Perkasie, PA (US)

(21) Appl. No.: 11/219,895

(22) Filed: Sep. 6, 2005

Related U.S. Application Data
(60) Provisional application No. 60/606,977, filed on Sep. 3, 2004.

(51) Int. Cl. A61B 17/10

(52) U.S. Cl. 606/139

(57) ABSTRACT

A tool for implantation of a flexible substrate into soft tissue is disclosed. The tool has a shaft with a bore. A penetrator tip is positioned at a distal end of the shaft. A handle is positioned at the proximal end. A push rod extends through the bore and is attached at one end to the penetrator tip, and at the other end to a trigger mounted on the handle. The penetrator tip is biased toward the shaft by a spring and captures the substrate between itself and the shaft. The shaft is inserted into soft tissue, drawing the substrate with it. Pulling the trigger releases the substrate from the tool. The shaft is withdrawn, leaving the substrate within the tissue.

### FIELD OF THE INVENTION

This invention relates to a tool for implanting an incontinence sling within the abdomen to treat urinary incontinence.

### BACKGROUND OF THE INVENTION

This invention relates to a tool for implanting an incontinence sling within the abdomen to treat urinary incontinence.

### SUMMARY OF THE INVENTION

The invention concerns a tool for implanting a flexible substrate within soft tissue, for example, an incontinence sling within the muscular tissue of the abdomen. The tool comprises an elongated shaft having a bore therethrough. A penetrator tip is positioned at one end of the shaft. The tip is movable toward and away from the shaft. A handle is positioned at an opposite end of the shaft, the handle facilitating manual grasping of the tool. A push rod is attached to the tip and extends through the shaft to the handle. A trigger is movably mounted on the handle and attached to the push rod. Motion of the trigger effects motion of the tip toward and away from the shaft by moving the push rod within the bore.

In use, a portion of the substrate is captured between the tip and the shaft by moving the tip toward the shaft with the portion of the substrate positioned therebetween. The shaft is manually insertable into the soft tissue thereby drawing the substrate into the tissue. Actuation of the trigger moves the tip away from the shaft and releases the substrate portion from the shaft. The shaft may then be withdrawn from the tissue leaving the substrate therein.

Preferably, the shaft is curved to permit it to be maneuvered around organs and bones within the abdomen. It is advantageous if the penetrator tip has a conical point to facilitate penetration into the soft tissue. The penetrator tip may engage the shaft within the bore to hold the substrate to the shaft. Preferably, a biasing member biases the penetrator tip toward the shaft. The biasing member may comprise a spring that engages either the trigger or the push rod and the shaft.

The invention also includes a method of implanting a flexible substrate in soft tissue. The method comprises the steps of:

(A) providing a tool having a shaft with a tip movable toward and away from the shaft;

(B) capturing the substrate between the tip and the shaft by moving the tip toward the shaft with the substrate positioned therebetween;

(C) inserting the shaft into the tissue, thereby drawing the substrate into the tissue;

(D) moving the tip away from the shaft thereby releasing the substrate from the tool; and

(E) withdrawing the shaft from the tissue while leaving the substrate therein.

### BRIEF DESCRIPTION OF THE DRAWINGS

FIG. 1 is a perspective view of a portion of the abdomen illustrating an implanted incontinence sling;

FIG. 2 is a side view of an incontinence sling having tunneling needles attached according to the prior art;

FIG. 3 is a median plane sectional view of the abdomen;

FIG. 4 is a longitudinal sectional view of an implantation tool according to the invention; and

FIG. 5-7 illustrate the use of the tool shown in FIG. 4 for the implantation of a flexible substrate within soft tissue.

### DETAILED DESCRIPTION OF THE EMBODIMENTS

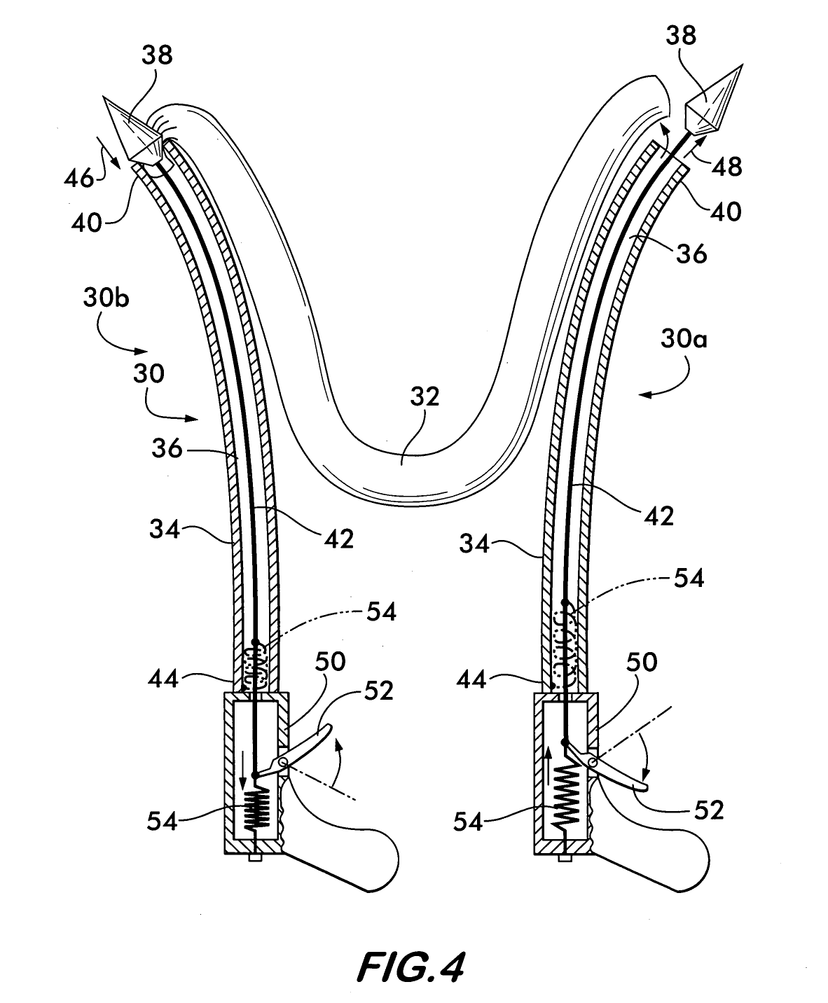

FIG. 4 shows a tool 30 that is adapted to implant a flexible substrate 32 within soft tissue. Substrate 32 may be, for example, an incontinence sling as described above, or another flexible material to be implanted within soft tissue. The substrate may be woven, knitted, braided, or a non-woven textile, as well as a polymer membrane or individual filamentary members. Tool 30 may be used singly or in pairs (30a and 30b) as shown depending upon the nature of the implantation procedure.

Tool 30 comprises an elongated shaft 34. The shaft is preferably stainless steel to provide the required strength and stiffness for insertion of the shaft through soft tissue. The stainless steel is also substantially inert, readily sterilized and compatible with living tissue. Shaft 34 has a bore 36 and is preferably curved to permit it to be maneuvered around organs, bones, and other structures within the body.

A penetrator tip 38 is positioned at a distal end 40 of shaft 34. Tip 38 preferably has a conical shape to facilitate penetration of the shaft into soft tissue. The tip may engage the shaft within the bore 36 for reasons explained below. The tip is attached to a push rod 42 that extends through the bore 36 to the proximal end 44 of the shaft. Tip 38 is movable toward and away from the shaft in response to motion of the push rod 42 as illustrated by arrows 46 and 48 in FIG. 4.

Proximal end 44 of shaft 34 is attached to a handle 50 that facilitates manual grasping and manipulation of tool 30. A trigger 52 is mounted on the handle 50, the trigger being movable and attached to push rod 42. Motion of the trigger effects motion of the pushrod to move the penetrator tip 38 toward and away from the distal end 40 of shaft 34 as illustrated by comparing the two tools 30a and 30b shown in FIG. 4. Preferably, tool 30 has a biasing member in the form of a spring 54 that biases the penetrator tip 38 toward the shaft 34. To effect the biasing action, spring 54 may engage the trigger 52 or it may engage the shaft 34 and the pushrod 42 as shown in phantom line.

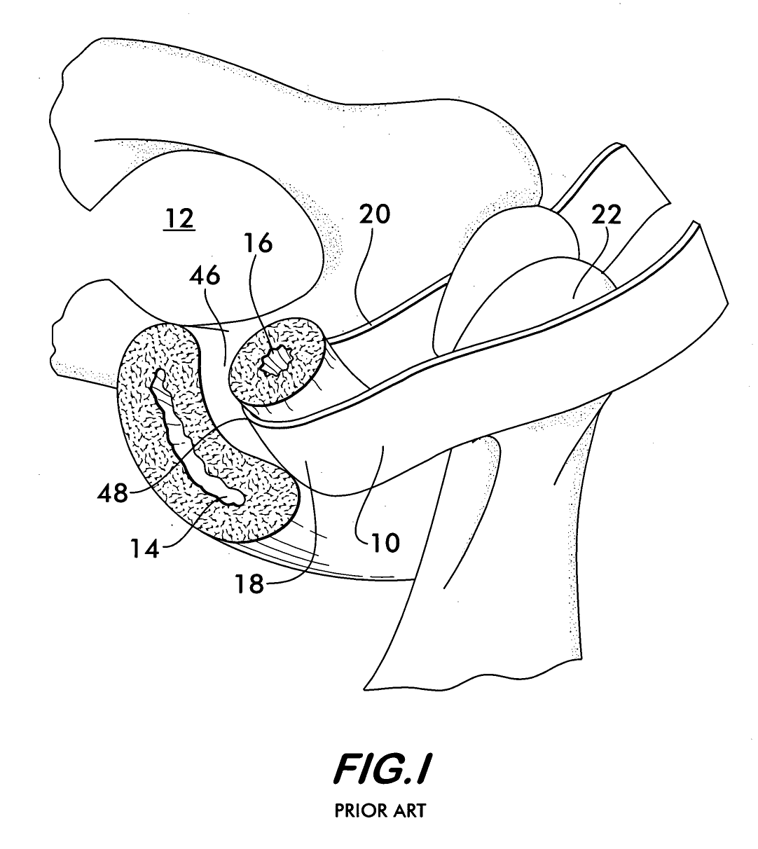

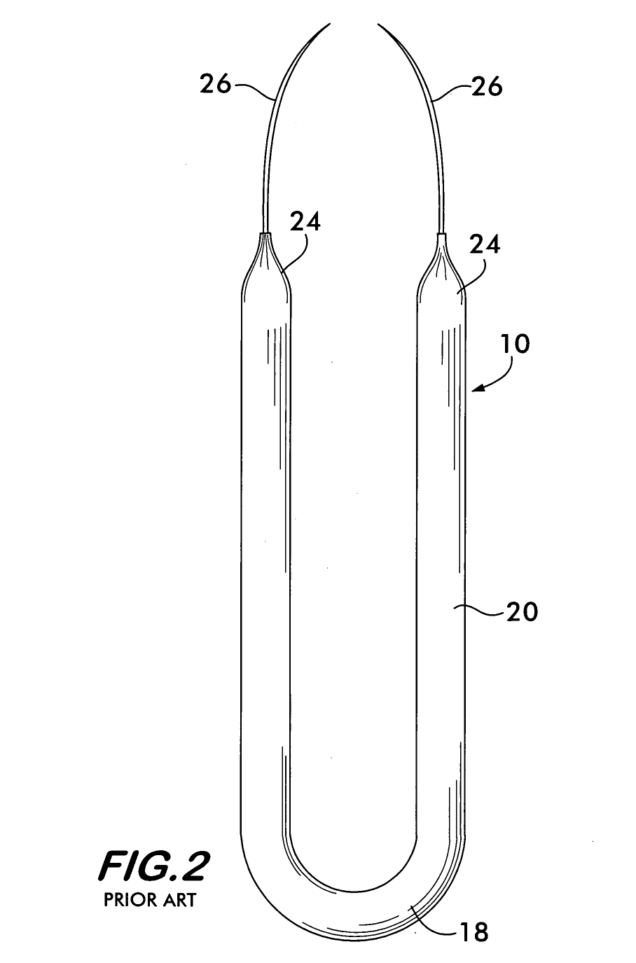

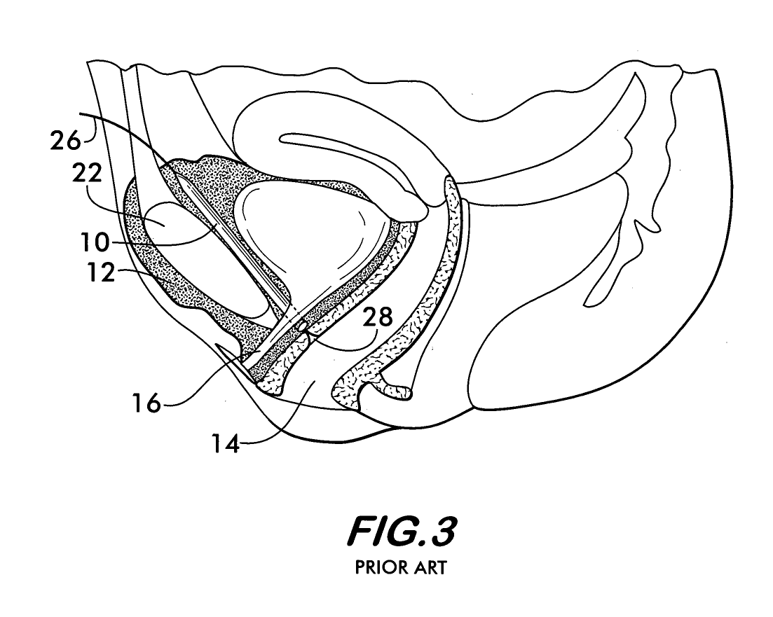

In operation, the flexible substrate 32 is captured between the penetrator tip 38 and the distal end 40 of shaft 34 by pulling trigger 52 to move the tip away from the shaft as shown at tool 30a of FIG. 1. A portion of the substrate is positioned between the tip and the distal end and the trigger is released, the spring 54 drawing the penetrator tip toward the shaft as shown at tool 30b. The tip and the shaft engage and hold the substrate 32 to the distal end of shaft 34. A secure grip on the substrate is achieved when the tip engages the shaft within the bore 36, this form of engagement providing increased contact area between the tip, the inside surface of the shaft and the substrate.

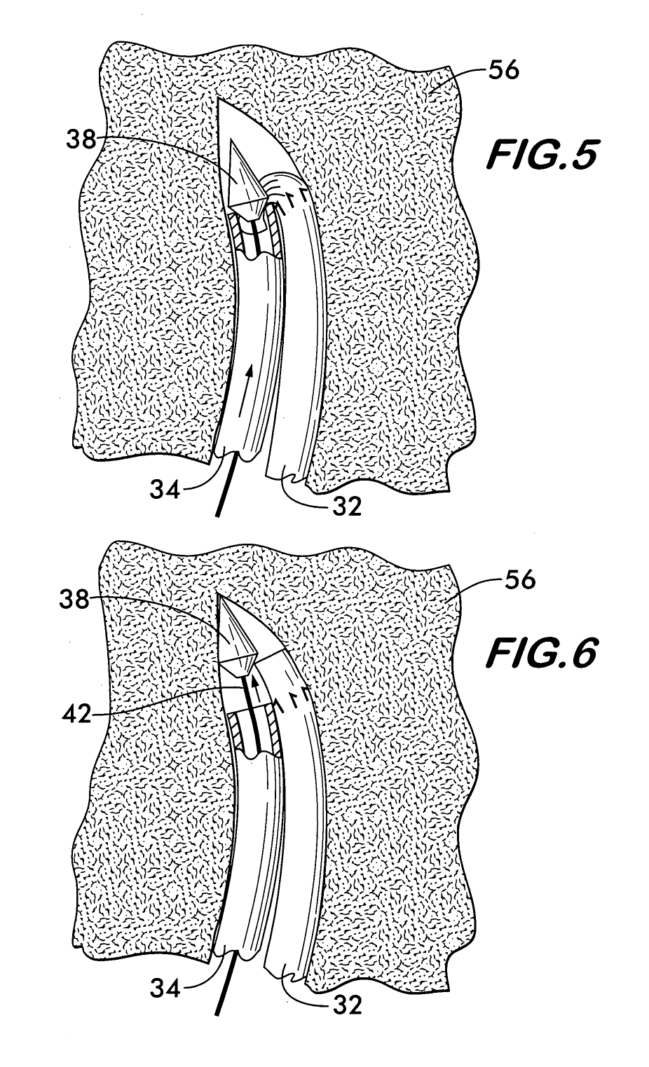

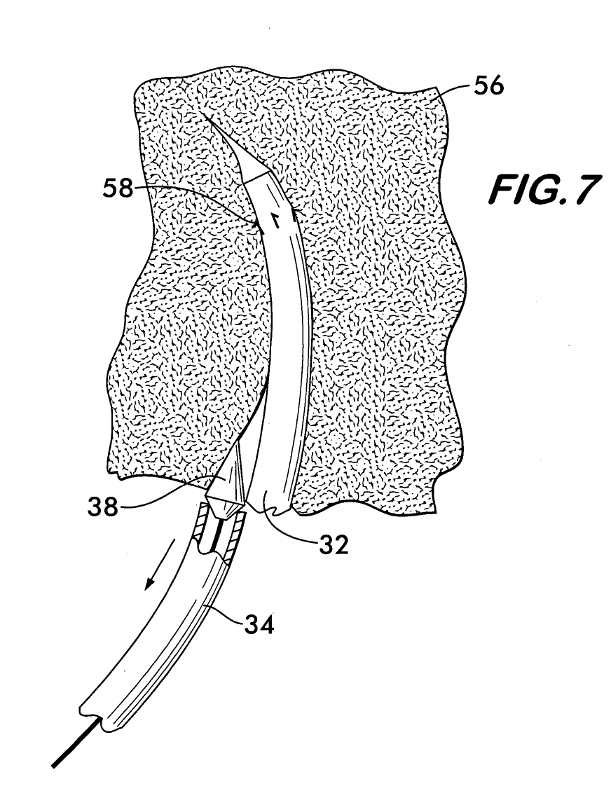

As shown in FIG. 5, the shaft 34 of the tool is then inserted into soft tissue 56 during a medical procedure, for example, the implantation of an incontinence sling. The penetrator tip 38 facilitates entry of the shaft into the tissue by virtue of its shape, and the substrate 32 is drawn into the tissue. When the desired degree of penetration is reached, as shown in FIG. 6, the substrate is released from the tool by moving the penetrator tip 38 away from the distal end 40 of shaft 34. This is effected by pulling trigger 52 (see FIG. 4) which moves the tip 38 through the action of push rod 42. The shaft 34 may then be withdrawn as shown in FIG. 7, leaving the substrate within the tissue 56 where it can be anchored to the tissue by hooks 58 attached to the substrate, or by intergrowth of the tissue with the substrate, as may conveniently occur for woven substrates wherein the cells of the tissue grow in the interstices formed by the woven filamentary members forming the substrate.

Tools for implantation of flexible substrates according to the invention reduce the trauma associated with such procedures because they limit the penetration of the tissue only to the degree that is necessary. This speeds the healing process and reduces the patient recovery time.

What is claimed is:

1. A tool for implanting a flexible substrate within soft tissue, said tool comprising:
an elongated shaft having a bore therethrough;
a penetrator tip positioned at one end of said shaft, said tip being movable toward and away from said shaft;
a handle positioned at an opposite end of said shaft facilitating manual grasping of said tool;
a push rod attached to said tip and extending through said shaft to said handle;
a trigger movably mounted on said handle and attached to said push rod, motion of said trigger effecting motion of said tip toward and away from said shaft by moving said push rod within said bore; and wherein:
a portion of said substrate being captured between said tip and said shaft by moving said tip toward said shaft with said portion of said substrate positioned therebetween, said shaft being manually insertable into said soft tissue thereby drawing said substrate into said tissue, actuation of said trigger moving said tip away from said shaft and releasing said substrate portion from said shaft, said shaft being withdrawn from said tissue leaving said substrate therein.

2. A tool according to claim 1, wherein said shaft is curved.

3. A tool according to claim 1, wherein said penetrator tip has a conical point.

4. A tool according to claim 1, wherein said penetrator tip engages said shaft within said bore to hold said substrate to said shaft.

5. A tool according to claim 1, further comprising a biasing member biasing said penetrator tip toward said shaft.

6. A tool according to claim 5, wherein said biasing member comprises a spring.

7. A tool according to claim 5, wherein said biasing member engages said trigger.

8. A tool according to claim 5, wherein said biasing member engages said push rod and said shaft.

9. A tool for implanting an incontinence sling within soft tissue of an abdomen, said tool comprising:
an elongated shaft having a bore therethrough;
a penetrator tip positioned at one end of said shaft, said tip being movable toward and away from said shaft;
a handle positioned at an opposite end of said shaft facilitating manual grasping of said tool;
a push rod attached to said tip and extending through said shaft to said handle;
a trigger movably mounted on said handle and attached to said push rod, motion of said trigger effecting motion of said tip toward and away from said shaft by moving said push rod within said bore;
a biasing member biasing said penetrator tip toward said shaft; and wherein:
a portion of said sling being captured between said tip and said shaft by moving said tip toward said shaft with said portion of said substrate positioned therebetween, said shaft being manually insertable into said soft tissue thereby drawing said substrate into said abdomen, actuation of said trigger moving said tip away from said shaft and releasing said sling portion from said shaft, said shaft being withdrawn from said tissue leaving said sling therein.

10. A tool according to claim 9, wherein said shaft is curved.

11. A tool according to claim 9, wherein said penetrator tip has a conical point.

12. A tool according to claim 9, wherein said penetrator tip engages said shaft within said bore to hold said substrate to said shaft.

13. A tool according to claim 9, wherein said biasing member comprises a spring.

14. A tool according to claim 9, wherein said biasing member engages said trigger.

15. A tool according to claim 9, wherein said biasing member engages said push rod and said shaft.

16. A tool according to claim 9, further comprising:
a second elongated shaft having a bore therethrough;
a second penetrator tip positioned at one end of said second shaft, said second tip being movable toward and away from said second shaft;
a second handle positioned at an opposite end of said second shaft facilitating manual grasping of said tool;
a second push rod attached to said second tip and extending through said second shaft to said second handle;
a second trigger movably mounted on said second handle and attached to said second push rod, motion of said second trigger effecting motion of said second tip toward and away from said second shaft by moving said second push rod within said bore;
a second biasing member biasing said second tip toward said second shaft; and wherein:
a second portion of said sling being captured between said second tip and said second shaft by moving said second tip toward said second shaft with said second portion of said substrate positioned therebetween, said second shaft being manually insertable into said soft tissue thereby further drawing said substrate into said abdomen, actuation of said second trigger moving said second tip away from said second shaft and releasing said second sling portion from said second shaft, said second shaft being withdrawn from said tissue leaving said sling therein.

17. A method of implanting a flexible substrate in soft tissue, said method comprising the steps of:
providing a tool having a shaft with a tip movable toward and away from said shaft;
capturing said substrate between said tip and said shaft by moving said tip toward said shaft with said substrate positioned therebetween;
inserting said shaft into said tissue, thereby drawing said substrate into said tissue;
moving said tip away from said shaft thereby releasing said substrate from said tool; and
withdrawing said shaft from said tissue while leaving said substrate therein.
